(19) United States
(12) Patent Application Publication
(10) Pub. No.: US 2002/0096182 A1
(43) Pub. Date: Jul. 25, 2002

(54) Method of evaluation and selection of absorbent article

(76) Inventors: Takayuki Hisanaka, Kagawa-ken (JP); Yuri Imamura, Kagawa-ken (JP)

Correspondence Address: Paula Campbell Evans, Palmer & Dodge, LLP, One Beacon Street, Boston, MA, 02108, US

(21) Appl. No.: 09/887,963

(22) Filed: Jun. 22, 2001

(30) Foreign Application Priority Data
Sep. 1, 2000 (JP) 265696/2000

(51) Int. Cl. A61B 19/00

(52) U.S. Cl. 128/898

(57) ABSTRACT

The present invention relates to a method of evaluating and selecting an absorbent article using the cardiac rate fluctuation of the wearer as a measure. The method enables evaluating and selecting absorbent articles such as disposable diapers and sanitary napkins conforming to the user's physiological and psychological requirements.

### TECHNICAL FIELD

The present invention relates to a method of evaluating and selecting absorbent articles such as disposable diapers and sanitary napkins. More particularly, the present invention relates to a method of evaluating and selecting an absorbent article most appropriate for physiological and psychological requirements of users based on fluctuation of the cardiac rate (heart rate) of the wearer.

### BACKGROUND ART

At present, absorbent articles such as disposable diapers, sanitary napkins, vaginal discharge liners, and breast milk pads are classified for sale in the market according to the dimensions (length, thickness, etc.), configuration, absorption amount, price, and the like.

For this reason, users select and purchase these absorbent articles according to the dimensions, configuration, absorption amount, price, and the like.

In addition, in the absence of expert sales personnel in shops, the purchasers have to select and purchase the articles displayed on the shelves according to the dimensions, configuration, absorption amount, and the like described on the packages.

For example, when buying a diaper for infants, the purchaser must select the articles according to the size or the body weight (e.g. the size for new-born babies, S, M, L, etc.) and the type classified according to the infant's ability to stand and walk or not (e.g. a type of assembling using tapes, or shorts type). When purchasing a diaper for adults, the waist size (S, M, L, etc.) and the form may be the measure for selection. Sanitary napkins are selected according to the amount of absorption and length (depending on the amount of vaginal discharge) and the form (with or without flaps).

Because the users usually purchase sanitary napkins themselves, they can select the articles most suited for their condition and favorite (tactile sense, form, absorption amount, etc.) through their experience. In addition, the sanitary napkin users can cope with troubles caused by the goods by themselves by receiving therapeutic measures in hospital, etc.

However, a purchaser is not usually the user in the case of diapers for infants or adults. Therefore, the goods are generally selected through the judgment of the purchaser, who is a care person or a helper, according to the size, leak-proof properties, etc. This tendency is particularly strong in the case of infants not possessing sufficient language and exercise capability or elderly persons with dementia symptoms.

Said selection is thus not necessarily in accord with the physiological or psychological requirements of the users. Selected goods may impart an unacceptable feeling of use (tactile sense, tightness, movability) and may result in externally induced diseases such as diaper rash (contact dermatitis) or may cause a psychic stress in the users. If such an absorbent article is continuously used, immune functions of the user may deteriorate due to a latent psychic stress.

Under such commercial circumstances, efforts of personnel involved in the development and manufacturing of absorbent articles may be directed to the purchaser's taste and convenience, and the development of product performance may not be in line with the user's requirements.

However, no method for correcting such a situation has conventionally been developed. There have been no appropriate measures used in the evaluation and selection of absorbent articles conforming to the user's comfort and tactile sensation.

### DISCLOSURE OF THE INVENTION

An object of the present invention is therefore to solve the above problems in the conventional technology. A specific object of the present invention is to provide a method of evaluating and selecting absorbent articles such as disposable diapers and sanitary napkins conforming to the user's physiological and psychological requirements. More specifically, an object of the present invention is to provide a method enabling developers and manufacturers of diapers to evaluate and select diapers closer to the user's physiological and psychological requirements such as wearing comfort and a method enabling purchasers to evaluate and select diapers more appropriate to the user's physiological and psychological requirements.

As a result of extensive studies with an objective of solving the above problems, the present inventors have found that absorbent articles can be evaluated and selected by using cardiac rate fluctuation, by which internal (autonomic nervous system) conditions of the wearer can be ascertained, as a measure. This finding has led to the completion of the present invention.

In the investigation of various measures which can objectively estimate physiological and psychological conditions of the diaper users, the present inventors paid attention to the cardiac rate fluctuation and found that the degree of comfort of diaper is related to the cardiac rate fluctuation of the wearer. Specifically, the inventors found that the cardiac rate fluctuation of the wearer of an absorbent article when the wearer feels comfortable differs from the cardiac rate fluctuation when he or she feels uncomfortable.

Therefore, the present invention relates to:

(1) A method of evaluating suitability of absorbent articles using the cardiac rate fluctuation of the wearer as a measure,

(2) A method of selecting an absorbent article suitable for a person wearing the same by using the cardiac rate fluctuation of the wearer as a measure,

(3) The method of (1) or (2) above, wherein the measure is a high frequency component (HF component) obtained by the frequency analysis of the interval (wave shape) between R waves (R-R interval) of an electrocardiogram.

(4) The method of (1) or (2) above, wherein the measure is a ratio (LF/HF) of the low frequency component (LF component) and the high frequency component (HF component) obtained by frequency analysis of the interval between R waves (R-R interval) of an electrocardiogram.

The present invention exhibits the special effect that an absorbent article satisfying physiological and psychological requirements of the wearer such as degree of comfort can be objectively and easily selected and evaluated by using the cardiac rate fluctuation of the wearer as a measure.

### BRIEF DESCRIPTION OF THE DRAWINGS

FIG. 1 is a drawing showing the effect by the type of diaper on the HF component.

FIG. 2 is a drawing showing the effect by the type of diaper on the LF/HF ratio.

### DETAILED DESCRIPTION OF THE INVENTION AND PREFERRED EMBODIMENT

The present invention will be explained in more detail in the following description, which is not intended to be limiting of the present invention.

The absorbent articles in the present invention include disposable diapers, sanitary napkins, vaginal discharge liners, and breast milk pads. In addition, the present invention can also be applied to underwear shorts for incontinence and the like.

A series of characteristic electrical potentials are generated in the heart muscle during heat pulsation. An electrocardiogram is a curve obtained by recording the electrical potentials. An electrocardiogram is constituted sine waves from 0.5 Hz to 100-200 Hz divided into several signals such as a P wave, an R wave, and a T wave. Heat pulsation appears to be generated regularly in a certain constant periodic cycle. However, examination of the time interval between pulsations, for example the interval of R waves (R-R interval, RRI), revealed that the cycle is not necessarily fixed, but fluctuates within a range of about 10-30%. This fluctuation is the cardiac rate fluctuation. The cardiac rate fluctuation has recently attracted attention in the evaluation of the autonomous neurological function and psychic stress produced by non-acclimatization of the autonomous neurological function. Specifically, the degree of cardiac rate fluctuation can be used in the evaluation of the autonomic nervous system of humans. Various methods for the analysis of cardiac rate fluctuation have been proposed. It is known that cardiac rate fluctuation has several components with a different frequency and each component reflects peculiar activity of the autonomic nervous system. Therefore, the application of the method of frequency analysis to cardiac rate fluctuation is effective.

When the frequency analysis of cardiac rate fluctuation is carried out by the fast Fourier transform (FFT) , two fluctuation components, that is, a low frequency component (LF component) of 0.05-0.15 Hz and a high frequency component (HF component) of 0.2-0.4 Hz, emerge in the power spectrum.

The HF component extracted from the cardiac rate fluctuation spectrum is known as a measure of the parasympathetic nerve. Evaluation of the balance between the sympathetic nerve and the parasympathetic nerve using the ratio of the LF component and the HF component extracted from the cardiac rate fluctuation spectrum has also been attempted.

The present inventors have conducted experiments based on the idea that it might be possible to evaluate and select an absorbent article using cardiac rate fluctuation as a measure, and found that the cardiac rate fluctuation of the wearer of an absorbent article is related to the degree of comfort property of the absorbent article. This finding has led to the completion of the present invention. Although the relationship between cardiac rate fluctuation and the autonomic nervous system, sympathetic nerve system, or parasympathetic nerve system has conventionally been known, the application of cardiac rate fluctuation to the evaluation or selection of the absorbent articles has been conceived for the first time by the present inventors.

The present invention will be described in detail below. When the frequency of cardiac rate fluctuation is analyzed by the fast Fourier transform, a low frequency (LF) component (0.05-0.15 Hz) and a high frequency (HF) component (0.2-0.4 Hz) emerge in the power spectrum. The numeric obtained by integrating the LF component and the HF component can be used in evaluating whether the sympathetic nerve is predominant due to tension or the parasympathetic nerve is predominant due to relaxation. The results are then used for the evaluation of the degree of comfort and the selection of absorbent articles. The LF component is a measure of the sympathetic nerve and the parasympathetic nerve, the HF component is a measure of the parasympathetic nerve, and the ratio LF/HF can be used as a measure of the balance of the sympathetic nerve and the parasympathetic nerve.

The method of measuring the cardiac rate fluctuation and the method of analyzing the measured data will be outlined below (see also “The Handbook of Human Factors and Ergonomics”, edited by The Japan Society of Physiological Anthropology, Instrumentation Research Department, Gihodoshuppan Co., Ltd., pages 263-268, 387-399; and “How to measure biological functions for beginners”, edited by Shojiro KATO, et al., Japan Publication Services, Inc., pages 87-99).

### Method of Measuring Cardiac Rate Fluctuation

1) Instrument

A conventional heart rate meter and electrocardiograph can be used for the measurement of cardiac beat and providing an electrocardiogram. For example, a portable heart rate meter such as Accurex Plus (manufactured by POLAR Co., Ltd.) and MAC Heart Rate Memory (manufactured by Vine Co.), a polygraph such as Polygraph RM-6000 (manufactured by Nihon Kohden Corporation) and Polygraph 360 (manufactured by NEC Medical Systems Co., Ltd.), or an electrocardiograph can be used.

2) Electrodes

A plate electrode made of silver-silver chloride is used as the leading electrode.

3) Measuring Method

(i) The skin surface to be attached the electrodes is cleaned with a cotton puff containing rubbing alcohol and the electrodes are attached with an electrode paste to the skin surface.

(ii) The potential of the skin surface corresponding to the heart activity of the human body is extracted and the voltage differences between the electrodes are amplified with an AC amplifier.

(iii) The output signals are sent to an A/D converter, and converted to digital signals, and the signals as data are stored in the media such as DAT, MO, etc.

### Method Of Data Analysis

The peak site of the R wave is detected from the digital waveform obtained by the cardiac rate fluctuation measurement, and the R-R interval data is obtained for each beat. The frequency of the resulting data of the cardiac rate fluctuation is analyzed by the fast Fourier transformation. The LF component (0.05-0.15 Hz) and HF component (0.2-0.4 Hz) in the power spectra are numerically integrated, and then LF component (power), the HF component (power), and the ratio LF/HF are calculated.

### Test Of Wearing Absorbent Article

Specifically, a subject wore an absorbent article with a controlled absorption amount and tactile sensation and his cardiac rate fluctuation was measured while controlling the temperature and humidity at the interface of the skin and the absorbent article, to evaluate and select the absorbent article using the measured cardiac rate fluctuation as a measure.

The relation between the cardiac rate fluctuation and the sensation of use of the absorbent article was investigated as follows.

(Absorbent Article)

The following two pants-type diapers are used as the absorbent articles.

TABLE 1

| | Rate of | Rewet | Absorbed | Retained |
|---|---|---|---|---|
| | absorption | amount | amount | water amount |
| | (sec) | (g) | (g/p) | (g/p) |
| Diaper I | 30.0 | 6.4 | 807.0 | 384.0 |
| Diaper II | 27.2 | 76.7 | 504.0 | 177.0 |

Note:

Rate of absorption: the time required for the total amount of 200 ml of a physiological saline solution added to the diaper to be absorbed through the top sheet.

Rewet amount: the amount of a physiological saline solution absorbed in a filter paper placed on the top sheet of a diaper under a load of 35 g/CM² five minute after 200 ml of a physiological saline solution is added.

Absorbed amount: the value determined by subtracting the weight of a diaper before absorbing water from the weight of the diaper that was dipped in a physiological saline solution for 30 minutes, removed from the solution, and allowed to stand under a load of 35 g/cm² for 20 minutes to let water escape. (g/p indicates the amount of water absorbed per one diaper.)

The amount of retained water: the value determined by subtracting the weight of a diaper before absorbing water from the weight of the diaper after absorbing water according to the above procedure of determining the amount of water absorption and removing the water by centrifugation at 150G for 90 seconds.

The diaper I is a sample representing a diaper exhibiting good performance in rate of absorption, rewet amount, amount of water absorption, and amount of retained water, whereas diaper II is a sample exhibiting poor performance in all of these tested items.

(Wearing Test Method)

Seven adult panelists performed the wearing test.

The panelists wore the diaper I or II in a room at 28° C. and 60% RH. After 25 minutes, simulated urine at 37° C. was discharged into the inside of the diaper using a silicon tube. The panelists continued to wear the diaper for 25 minutes.

(Wearing Test Results)

The cardiac rate fluctuation before and immediately after discharge of simulated urine was determined for each panelist according to the above method and the frequency was analyzed using the above-described method to determine the HF component and the ratio LF/HF.

The average of the results of the seven panelists is shown in Table 2.

TABLE 2

| | HF Component | LF/HF Ratio |
|---|---|---|
| | immediately after urination | immediately after urination |
| Diaper I | 3.40 | 0.90 |
| Diaper II | 2.51 | 1.36 |

The results shown in Table 2 indicate that the average HF component after urination of the seven panelists was 3.40 for the diaper I with good performance, whereas the value was 2.51 for the diaper II with poor performance.

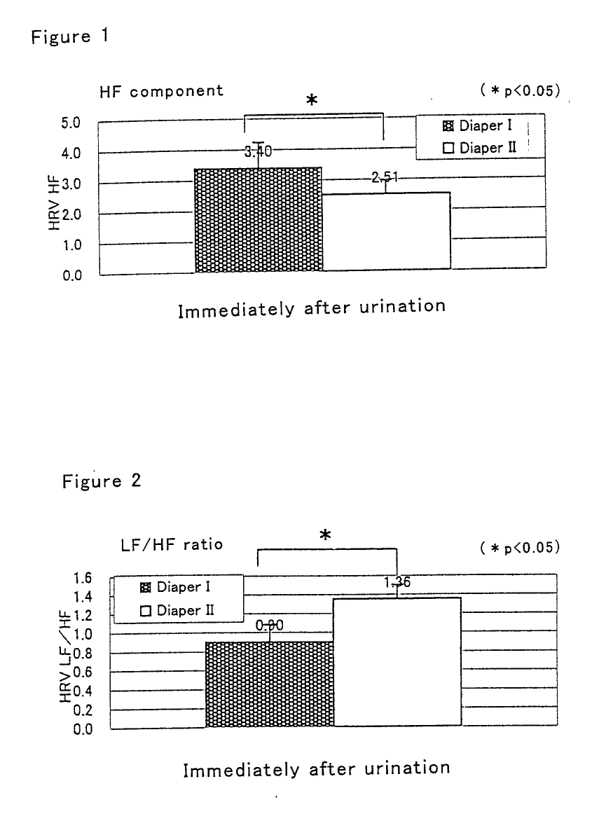

The HF component and the ratio LF/HF of Table 2 are diagrammatically shown in FIGS. 1 and 2 respectively.

FIG. 1 graphically shows the average HF component in diaper I and diaper II immediately after urination. FIG. 2 graphically shows the average LF/HF component in diaper I and diaper II immediately after urination.

Comparison of the diaper I and diaper II according to the significant difference verification (p<0.05) confirms that the diaper I shows a significantly higher HF component and a significantly lower LF/HF ratio after urination. This indicates that the wearer of diaper I feels more relaxed due to a predominant parasympathetic nerve, whereas the wearer of diaper II feels more strained due to a predominant sympathetic nerve.

As a result, a diaper with good performance is judged to exhibits a high HF component and a low LF/HF value. Therefore, it is possible to evaluate and select a diaper suitable for a wearer by measuring his or her HF component and/or LF/HF ratio.

These results show that the measures representing the cardiac rate fluctuation (the HF component and LF/HF ratio) are not only usable as a measure for evaluating diapers in the development and manufacture, but also applicable as a measure for selecting diapers imparting a comfortable and good wearing feeling to users such as infants and aged persons who cannot express their own feelings.

More specifically, when the HF component is high and/or the LF/HF ratio is low, the diaper is judged to be good. If these results are applied, it is possible to provide diapers more appropriate to the users (wearers) by developing and manufacturing diapers exhibiting a high HF component and a low LF/HF ratio.

The present invention has been described referring to the relationship between the absorption performance of a diaper and the cardiac rate fluctuation in the above description, an absorbent article most appropriate for the user in terms of other properties such as tactile sense, tightness, and movability can also be selected by determining the cardiac rate fluctuation in the same manner.

In addition, although the relationship between the absorbent article and the cardiac rate fluctuation is determined using only diaper as an example of the absorbent article in the above description, the same results can also be predicted for other absorbent articles.

### EXAMPLES

The present invention will now be described by way of examples, which should not be construed-as limiting the present invention.

Three types of diapers I-III shown below were used for the wearing test by aged persons virtually requiring care. The absorption properties of the three diapers I-III are shown in Table 3.

TABLE 3

| | Rate of | Rewet | Absorbed | Retained |
|---|---|---|---|---|
| | absorption | amount | amount | water amount |
| | (sec) | (g) | (g/p) | (g/p) |
| Diaper I | 30.0 | 6.4 | 807.0 | 384.0 |
| Diaper II | 27.2 | 76.7 | 504.0 | 177.0 |
| Diaper III | 28.6 | 43.6 | 638.0 | 256.0 |

The same methods of measurement of the rate of absorption, rewet amount, absorption amount, and retained water amount as in the Table 1 were applied.

1) Cardiac Rate Fluctuation And The Degree Of Comfort

The HF component and the LF/HF ratio measured for seven panelists immediately after urination in the wearing test are shown in Table 4.

Degree of comfort felt by the wearers after urination was evaluated based on the following criteria of three levels.

3: Almost no change in the wearing sensation after urination as compared with before urination.

2: The wearing sensation was slightly uncomfortable after urination as compared with before urination.

1: The wearing sensation was uncomfortable after urination as compared with before urination.

The values averaged for the seven panelists are also shown in Table 4.

2) Observation Of Skin Conditions

Aged persons requiring care wore the diapers, which were replaced seven times a day on average. After five days, the skin conditions were observed. The results are also shown in Table 4.

TABLE 4

| | HF Component | LF/HF Ratio | | |
|---|---|---|---|---|
| | immediately | immediately | | |
| | after | after | Degree of | Condition |
| | urination | urination | comfort | of the skin |
| Diaper I | 3.40 | 0.90 | 2.7 | No |
| | | | | abnormalities |
| Diaper II | 2.50 | 1.36 | 1.1 | erythema |
| Diaper III | 2.76 | 1.04 | 1.9 | Slight |
| | | | | erythema |

The results of Table 4 confirm that the HF component and/or LF/HF ratio in the cardiac rate fluctuation are related to the degree of comfort, and also to the skin conditions of the wearer when he continuously wears the diaper. Specifically, it can be understood that the diaper having high HF component of the wearer is excellent on the degree of comfort and the skin conditions; and the diaper having low LF/HF ratio of the wearer is excellent on the degree of comfort and the skin conditions.

### INDUSTRIAL USEFULNESS

The use of the cardiac rate fluctuation as a measure of evaluation according to the present invention ensures objective evaluation of an absorbent article possessing appropriate functions. The present invention therefore can be applied to development of an absorbent article possessing appropriate functions. In addition, an absorbent article satisfying the wearer's desired degree of comfort can be selected and purchased by determining the wearer's cardiac rate fluctuation comprising the HF component and LF/HF ratio.

1. A method of evaluating suitability of absorbent articles using the cardiac rate fluctuation of the wearer as a measure.

2. A method of selecting an absorbent article suitable for a person wearing the article by using the cardiac rate fluctuation of the wearer as a measure.

3. The method according to claim 1 or 2, wherein the measure is a high frequency component (HF component) obtained by the frequency analysis of the interval between R waves (R-R interval) of an electrocardiogram.

4. The method according to claim 1 or 2, wherein the measure is a ratio (LF/HF) of the low frequency component (LF component) and the high frequency component (HF component) obtained by the frequency analysis of the interval between R waves (R-R interval) of an electrocardiogram.
